(19) United States
(12) Patent Application Publication
(10) Pub. No.: US 2014/0311941 A1
(43) Pub. Date: Oct. 23, 2014

(54) PACKAGE SYSTEM FOR STERILIZING AND STORING CYANOACRYLATE ADHESIVE COMPOSITIONS

(71) Applicant: ADHEZION BIOMEDICAL, LLC, Wyomissing, PA (US)

(72) Inventors: Sheng Zhang, Hickory, NC (US); Rafael Ruiz, SR., Hudson, NC (US)

(73) Assignee: Adhezion Biomedical, LLC, Wyomissing, PA (US)

(21) Appl. No.: 13/866,131

(22) Filed: Apr. 19, 2013

(51) Int. Cl. A61J 1/06

(52) U.S. Cl. CPC A61J 1/067
USPC 206/530

(57) ABSTRACT

Package systems that provide for prolonged shelf stability of sterilized cyanoacrylate compositions are provided. The package systems include an ampoule made from a material that is substantially impervious to gas and moisture penetration that includes a frangible foil seal, and an overpack. The ampoule contains a stabilized cyanoacrylate monomer composition, and may be sterilized by irradiation without substantially altering the viscosity of the composition. The package may be sterilized by ethylene oxide without substantially further altering the viscosity of the composition. The sterilized composition, when contained within the package system, is shelf-stable for a period of at least 24 months.

### FIELD OF THE INVENTION

The invention relates generally to the field of medical adhesives, and in particular, relates to packaging systems for storing and sterilizing cyanoacrylate monomer compositions, while maintaining suitable viscosity of the composition over an extended shelf life of at least two years.

### BACKGROUND OF THE INVENTION

Various publications, including patents, published applications, technical articles and scholarly articles are cited throughout the specification. Each of these cited publications is incorporated by reference herein, in its entirety and for all purposes.

As excellent adhesives, cyanoacrylate compositions have found wide applications as industrial and structural adhesives, consumer products for repair of household items and in the hobby sector for assembly and repair of models. More recently, liquid cyanoacrylate compositions have been used as medical adhesive for closing wounds and incisions, especially in cases where suturing does not provide satisfactory results, because of their unique ability to bond living tissue and their long-term bond strength.

Many packaging materials are available for cyanoacrylate compositions, but these materials are generally used for industrial adhesives. To be used in the medical fields, such as the surgical adhesive and microbial sealant drape, cyanoacrylate compositions must be sterile.

Sterilization of cyanoacrylate compositions can be accomplished by common techniques such as heat sterilization, ethylene oxide sterilization (ETO), microwave sterilization, UV light sterilization, gamma irradiation and electron beam sterilization. Sterilization according to such methodologies, however, suffer from serious drawbacks. High temperatures required for the dry heat sterilization processes often cause premature polymerization of the cyanoacrylate monomers. The toxic and explosive nature as well as the ineffective sterilization of ETO prevents the extensive application of ETO on the sterilization of cyanoacrylate compositions. In addition, the steam that typically accompanies ETO sterilization introduces water to the monomer, and water readily induces polymerization of the monomers. High doses of radiation will cause changes in the cyanoacrylate adhesive compositions as well, including polymerization and degradation.

Attempts to minimize sterilization-induced detrimental changes have focused on the addition of very high levels of polymerization inhibitors. Such high levels of inhibitors increase the toxicity of the mixture and increase the toxic by-products formed upon gamma irradiation. The use of high doses of toxic gamma irradiation to effect sterilization also raises safety concerns for workers who are exposed long term to this radiation. Electon beam or E-beam sterilization of cyanoacrylates often leads to a reduced shelf life, which is not desirable for a medical adhesive.

In spite of the challenges described above, irradiation sterilizations are advantageous over conventional sterilization techniques. The sterilizing effect of radiation is instantaneous and simultaneous in the whole of the target. The chemical reactivity of irradiation is relatively low compared to the often highly reactive gases involved in the chemical reactions. Irradiation leads to an insignificant rise in temperature, and irradiation easily reaches all parts of the objects to be sterilized. Irradiation can sterilize materials in their final packages, which provides considerable flexibility in packaging for sterilization and allows the product to be retained in the sterile form until the package is opened or damaged.

Nonetheless, it is well-known that irradiation sterilization can have a drastic and negative effect on the stability and performance of cyanoacrylate compositions. As specified in U.S. Publ. No. 2008/0021139, the exposure of cyanoacrylate esters to E-beam radiation can result in a partial polymerization of the monomers thus affecting both the shelf life and the performance of the cyanoacrylates. U.S. Pat. No. 6,248,800 describes the sterilization of cyanoacrylate adhesives using E-beam radiation. The exposure of cyanoacrylate esters to E-beam radiation, however, can result in a partial polymerization of the monomers thus affecting both the shelf life and the performance of the cyanoacrylates. In addition, U.S. Publ. No. 2005/0197421 describes that the sterilization conditions can be selected such that the cyanoacrylate ester undergoes partial polymerization to reactive oligomers having a higher viscosity. Incorporation of polymerization initiator into cyanoacrylates could make the sterilization process even more challenging since it leads to a higher degree of partial polymerization or full polymerization of adhesives upon irradiation sterilization.

In order to overcome the potential challenges induced by the polymerization accelerator during the sterilization process, different approaches have been employed, including the separation of the polymerization accelerators from the cyanoacrylate monomer during the sterilization process. For example, U.S. Publ. Nos. 2005/0047846 and 2007/0078207, and U.S. Pat. No. 6,579,469 reveal that the polymerization accelerators are applied to the applicator tip, or to be coated on an interior surface of the applicator body, or on an exterior surface of an ampoule or other container disposed within the applicator body. U.S. Publ. Nos. 2010/0330027, 2010/0269749, and 2008/0241249, and U.S. Pat. Nos. 6,620,846 and 5,928,611 describe an applicator tip having a polymerization or cross-linking initiator or accelerator disposed on or in a solid support in the applicator tip, wherein the cyanoacrylate monomer is located in the container body in a non-contacting relationship with the tip prior to dispensing the material.

Incorporation of polymerization accelerators into the applicator tip, however, is a complicated process since the polymerization accelerator has to either be applied as a solid coating onto the applicator tip by vapor deposition such as by sputtering, or be incorporated into the applicator tip by mixing the accelerator with the applicator tip material prior to molding. It is difficult to uniformly distribute the polymerization accelerator onto the applicator tip via such processes. It is also hard to control the amount of accelerator dissolved into the adhesive composition passing through the applicator tip. It can lead to clogging of the applicator if too much accelerator is incorporated resulting in an extremely fast curing. On the other hand, the accelerating effect may not be achieved if too little accelerator is incorporated when the adhesive flows through the applicator tip.

In order to overcome the challenges related to sterilizing cyanoacrylates by irradiation methods and fully benefit from the advantages of the irradiation technique, it is desirable to have a suitable package system where the cyanoacrylate adhesive composition can be sterilized in the absence or presence of miscible polymerization accelerator without having to separate the polymerization accelerator from the cyanoacrylate adhesive composition.

### SUMMARY OF THE INVENTION

The invention provides for suitable packages for sterilizing cyanoacrylate compositions via irradiation methods, such as E-beam, Gamma, or X-ray sterilization. The package comprises a primary package and a secondary overpack. The primary package may include a plastic ampoule and a multi-layer foil seal. The secondary overpack may include a front wrapper and a back wrapper. The primary package may be constructed with gas/moisture resistible materials. The plastic ampoule and the multi-layer foil seal may be heat-sealed together to form a container at elevated temperature and pressure to ensure a leak-free environment. Part of or the entire secondary overpack may be constructed with a gas-permissible material so that vapor and/or gas can penetrate the secondary overpack. The package body, as the delivery system of the stable cyanoacrylate compositions, may be constructed as bottles, applicators, vials, syringes, ampoules, or the like.

According to one embodiment, the invention provides a package system including a stabilized cyanoacrylate composition that is sterilized twice. The package system includes a stable cyanoacrylate composition having a viscosity (e.g., ranging from about 1-400 centipoise or cPs), an ampoule, and a secondary overpack. The ampoule contains the stable cyanoacrylate composition sealed with a multi-layer foil seal. The ampoule is made from an oxygen and/or moisture impermeable material. The ampoule is sterilized by irradiation while maintaining the viscosity of the stable cyanoacrylate composition such that a change in the viscosity is no more than 20 cPs. The secondary overpack includes a gas permeable back wrapper housing the ampoule containing the stable cyanoacrylate composition. The secondary overpack is sterilized by chemical sterilization while maintaining the viscosity of the stable cyanoacrylate composition such that a change in the viscosity is no more than 20 cPs. Thus, the package system provides a sterilized and stable cyanoacrylate composition with a shelf life of at least 12 months, preferably at least 24 months.

According to another embodiment, the invention provides for a method for sterilizing and storing cyanoacrylate compositions in the absence or presence of a polymerization accelerator. The method for sterilizing cyanoacrylate compositions in a package system includes (1) preparing cyanoacrylate monomer(s) with a purity of about 97-99% by weight; (2) stabilizing cyanoacrylate compositions with free radical and anionic polymerization inhibitors and dissolving polymerization accelerator in the cyanoacrylate compositions; (3) filling and sealing the cyanoacrylate compositions into a primary package; (4) sterilizing the cyanoacrylate compositions in the primary package system via an irradiation method; (5) assembling the primary package into an applicator with an applicator tip and packing into a secondary overpack; and (6) sterilizing the whole package system via a chemical sterilization.

The cyanoacrylate compositions are preferably highly pure, for example, on the order of about 98% purity. The high purity of cyanoacrylate monomer may be obtained, for example, by multiple distillations under high vacuum and high temperature. The cyanoacrylate compositions may contain free radical and anionic stabilizers. A trace amount of polymerization accelerator may be dissolved in the cyanoacrylate compositions before storing in the package systems.

The package system may include a plastic container and a multi-layer foil seal. The plastic container may be heat-sealed by the multi-layer foil seal after the adhesive is filled into the container. The temperature used to seal the plastic ampoule and the seal foil may be in the range of about 100° C. to about 200° C., preferably about 100° C. to about 180° C., and more preferably about 110° C. to about 170° C. The pressure used to seal the plastic container and the multi-layer seal foil may be in the range of about 1 bar to about 50 bar, preferably about 1 bar to about 40 bar, more preferably about 1 bar to about 25 bar, and most preferably about 1 bar to about 15 bar.

The invention provides for a suitable package for storing and sterilizing cyanoacrylate adhesive compositions wrapped with a secondary overpack. The secondary overpack may include a front wrapper and a back wrapper. The secondary overpack materials are preferably compatible with radiation or ETO sterilization methods.

One advantage of the invention is the selection of suitable package systems for cyanoacrylate adhesive compositions, which are compatible with the irradiation methods such as E-beam, Gamma, or X-ray sterilization to effectively sterilize the cyanoacrylate monomers inside. The components of the package are stable upon high dosages of irradiation. The packages provide a desired barrier to moisture so that premature polymerization of the sterilized cyanoacrylate monomer can be inhibited and prevented.

The irradiation methods were found to have a negligible effect on the performance of the cyanoacrylate adhesive compositions stored in the packages described herein. It was found that viscosity and set time of different cyanoacrylate compositions may only vary slightly upon sterilization methods, indicating that irradiation techniques such as E-beam, Gamma, or X-ray sterilization are compatible with the packaging to provide sterile and stable cyanoacrylate monomer compositions.

The packages for storing the cyanoacrylate adhesive compositions may include a small amount of one or more stabilizers. Large amounts of stabilizers can increase the toxicity of the cyanocrylate adhesive compositions. The cyanoacrylate compositions, as packaged, can be effectively sterilized via E-beam, Gamma, or X-ray irradiation in the presence of smaller amounts of anionic stabilizers, such as 50 ppm or less. The barrier property of the packages are suitable for the irradiation sterilization methods, which provides the sterility and long term stability of the cyanoacrylate adhesive compositions even in the presence of a small amount of stabilizer(s).

The package for storing and sterilizing the cyanoacrylate adhesive compositions via irradiation methods help to provide for an extended shelf life of the cyanoacrylate adhesive compositions. For example, the shelf life may be on the order of 12 months or more, preferably at least 24 months. The shelf life stability of the cyanoacrylate compositions with small amounts of stabilizers in the packaging system, as evaluated by the accelerated aging shelf life study (e.g., accelerated aging tests at 55° C. for 85 days and at 80° C. for 13 days) as well as the real time shelf life study, confirms an extended shelf life of at least 2 years of the cyanoacrylate adhesive compositions in the package after the irradiation sterilization.

### DETAILED DESCRIPTION OF THE INVENTION

In order to overcome the challenges related to stabilizing and sterilizing cyanoacrylates and take advantage of the benefits of irradiation sterilization, the processes, formulations, and packages for sterilizing stable cyanoacrylate compositions were developed incorporating irradiation. To maintain the stability of cyanoacrylate compositions and get extended shelf life of the cyanoacrylate adhesives, the careful selection of the packaging materials and the delicate design of the container were considered. The packaging materials for the cyanoacrylate adhesives should be essentially inert to the cyanoacrylate compositions during the period in which the composition is contained therein, for example, the package materials should not induce or facilitate polymerization of the cyanoacrylate monomers of the composition, and should shield the monomers from environmental conditions, including the presence of moisture, that may induce premature polymerization such that the compositions may enjoy extended shelf stability.

The invention provides for a package system for stable cyanoacrylate compositions that is compatible with both irradiation and chemical sterilization methodologies. The package system for the irradiation sterilization of cyanoacrylate composition also provides the sterile cyanoacrylate adhesive with an extended shelf life of at least one year, preferably at least two years. The package systems can also be used to house, sterilize, and store cyanoacrylate compositions that include a polymerization accelerator mixed together with the cyanoacrylate monomers, including sterilization via irradiation, while maintaining shelf stability without a substantial increase in viscosity of the sterilized composition when stored for two years or when maintained in the package system at 80° C. for 12 to 13 days.

The terms “comprising” and “including” are inclusive or open-ended and do not exclude additional unrecited elements, compositional components, or method steps. Accordingly, the terms “comprising” and “including” encompass the more restrictive terms “consisting essentially of” and “consisting of.” Unless specified otherwise, all values provided herein include up to and including the endpoints given, and the values of the constituents or components of the compositions are expressed in weight percent or % by weight of each ingredient in the composition.

### Package System

In some aspects, the invention provides a package system including a stabilized cyanoacrylate monomer composition. The composition is preferably sterilized twice. The package system includes a stabilized cyanoacrylate monomer composition having a viscosity, a primary package or ampoule, and a secondary overpack into which the ampoule is housed. The primary package or ampoule contains the stable cyanoacrylate composition in a chamber that has been sealed with a multi-layer foil seal. The primary package or ampoule may be made from a gas and/or moisture impermeable material. Preferably, the material is impermeable to oxygen and water vapor. The primary package or ampoule, which houses the stabilized cyanoacrylate monomer composition can be sterilized by irradiation while substantially maintaining the viscosity of the composition such that the composition does not experience a significant increase in viscosity following irradiation sterilization. The secondary overpack includes a gas-permeable wrapper that surrounds and houses the primary package or ampoule containing the stable cyanoacrylate composition. The secondary overpack can be sterilized by chemical sterilization, while substantially maintaining the viscosity of the stable cyanoacrylate composition such that the composition does not experience a significant increase in viscosity following sterilization. Thus, the package system is twice sterilized and provides a stable cyanoacrylate composition that has a shelf life of at least 12 months, preferably at least 24 months.

### Stable Cyanoacrylate Composition

The stable cyanoacrylate composition or cyanoacrylate adhesive may be comprised of cyanoacrylate monomers. The cyanoacrylate compositions are stable in that the compositions do not deteriorate, degrade, polymerize, react, form by-products, or otherwise break down or change the properties of the composition. The cyanoacrylate monomers may be synthesized by following procedures known in the art, for example, as described in U.S. Pat. Nos. 4,364,876, 2,721,858 and 3,254,111. For example, the cyanoacrylates may be prepared by reacting cyanoacetate with formaldehyde in the presence of a basic condensation catalyst at a high temperature to give a low molecular weight polymer. A depolymerization step followed under a high temperature and a high vacuum in the presence of acidic and anionic inhibitors, yields a crude monomer that can be distilled under the high vacuum in the presence of radical and acidic inhibitors, for example.

The cyanoacrylate monomers may comprise any cyanoacrylate monomers suitable in the art for adhesive applications, particularly for medical adhesive applications. For example, the cyanoacrylate monomer may comprise cyanoacrylate ester monomers. More specifically, the cyanoacrylate monomer may be an aliphatic cyanoacrylate ester and preferably an alkyl, cycloalkyl, alkenyl, alkoxyalkyl, fluororoalkyl, fluorocyclic alkyl or fluoroalkoxy cyanoacrylate ester. The alkyl group may contain from 2 to 12 carbon atoms, is preferably a C₂ to C₁₀ alkyl ester, and is most preferably a C₄ to C₈ alkyl ester. Suitable cyanoacrylate esters include without limitation, the ethyl, n-propyl, iso-propyl, n-butyl, pentyl, hexyl, cyclohexyl, heptyl, n-octyl, 2-ethylhexyl, 2-methoxyethyl and 2-ethoxyethyl esters. Any of these cyanoacrylate monomers may be used alone, in combination, or they may be used as mixtures. 2-octel cyanoacrylate monomer, as well as 2-octyl cyanoacrylate monomer mixed together with n-butyl cyanoacrylate monomer are preferred for the compositions.

The cyanoacrylate monomers may be of high purity. In other words, the cyanoacrylate monomers contain little to no impurities or contaminants. The purity of cyanoacrylate may be at least about 97% by weight, preferably at least about 98% by weight, and more preferably at least about 99% by weight. The purity of cyanoacrylate monomer may be obtained through one or more processes known in the art. In an exemplary embodiment, the high purity cyanoacrylate monomers may be obtained through a distillation process. For example, the high purity of cyanoacrylate monomer may be obtained by multiple distillations under high vacuum and high temperature. The vacuum for distilling cyanoacrylate monomer may be in the range of about 0.02 Torr to about 15 Torr, preferably in the range of about 0.05 Torr to about 10 Torr, and more preferably in the range of about 0.1 Torr to about 10 Torr. The distillation temperature may be in the range of about 100° C. to about 180° C., preferably in the range of about 100° C. to about 160° C., and more preferably in the range of about 100° C. to about 150° C. The distilled cyanoacrylate monomers may be formulated with free radical and acidic inhibitors depending upon their application and stability.

As will be recognized in the art, basic polymers or copolymers may be applied to reduce the amount of contaminants and extraneous additives in the cyanoacrylate monomer, but this can lead to several problems including premature polymerization. Some basic polymers or copolymers are not soluble in cyanoacrylate but are mixed with the monomer adhesive in mutual contact until the adhesive is destabilized. In order to achieve the mutual contact, such polymers or copolymers are mixed with the cyanoacrylate monomer under vacuum for a minimum of 3 hours, which may remove possible acid residues to destabilize the adhesive. The solid powder of such polymer is then removed from cyanoacrylate adhesive by filtering, for example, through a 0.2 μm filter.

The distilled or undistilled cyanoacrylate monomers can be filtered through one or multiple filters in order to reduce the bioburden level of the cyanoacrylate composition and remove any immiscible impurities or contaminants. If filtered, the cyanoacrylate monomers may be filtered through any suitable sized filters known in the art. For example, in a multiple step filtration process, the cyanoacrylate monomers may be filtered through a primary filter and one or more additional or secondary filters. The size of the primary filter may range, for example, on the order of about 0.01 to about 0.8 μm, preferably in the range of about 0.01 to about 0.6 μm, and more preferably in the range of about 0.03 to about 0.6 μm. The size of the additional or secondary filters may range, for example, on the order of about 1 to about 200 μm, preferably in the range of about 1 to about 150 μm, and more preferably in the range of about 1 to about 100 μm.

Viscosity includes the resistance of a fluid to flow due to a shearing force. The viscosity may be dependent upon the conditions under which it is measured, such as fluid temperature. Unless indicated otherwise, the absolute viscosity may be determined under standard temperature and pressure (i.e., 25° C. and atmospheric pressure) and is expressed in units of centipoise (cPs).

The stabilized cyanoacrylate monomer compositions, which may include one or more of a polymerization accelerator, a thickening agent, or a plasticizing agent, among any other additives described or exemplified herein, comprise an initial viscosity or first viscosity. This initial or first viscosity is the viscosity of the composition with all of its constituents mixed together, without being sterilized. The initial or first viscosity may be less than about 400 cPs, less than about 300 cPs, less than about 200 cPs, less than about 100 cPs, less than about 50 cPs, less than about 25 cPs, less than about 20 cPs, less than about 15 cPs, less than about 10 cPs, or less than about 7 cPs. In particular, the initial viscosity of the cyanoacrylate composition may be in the range of about 3 cPs to about 100 cPs, about 3 cPs to about 50 cPs, about 3 cPs to about 20 cPs, about 3 cPs to about 10 cPs, about 4 cPs to about 15 cPs, about 4 cPs to about 8 cPs, about 5 cPs to about 10 cPs, about 5 cPs to about 7 cPs, about 5 cPs to about 9 cPs, about 5 cPs to about 8 cPs, about 5 cPs to about 100 cPs, about 5 cPs to about 50 cPs, about 5 cPs to about 20 cPs, about 5 cPs to about 15 cPs, about 10 cPs to about 20 cPs, about 10 cPs to about 25 cPs, about 6 cPs to about 7 cPs, about 6 cPs to about 8 cPs, about 6 cPs to about 10 cPs, about 7 cPs to about 10 cPs, about 7 cPs to about 9 cPs, about 10 cPs to about 50 cPs, about 10 cPs to about 20 cPs, about 15 cPs to about 20 cPs, about 15 cPs to about 25 cPs, about 10 cPs to about 30 cPs, about 20 cPs to about 25 cPs, about 20 cPs to about 30 cPs, about 25 cPs to about 75 cPs, about 25 cPs to about 50 cPs, or about 25 cPs to about 30 cPs, prior to sterilization. Cyanoacrylate compositions containing a thickening agent and/or polymerization accelerator may have higher viscosities than compositions with only a thickening agent or a polymerization accelerator or compositions with neither a thickening agent or polymerization accelerator.

Various additives can be mixed together with the cyanoacrylate monomers as part of the cyanoacrylate compositions. Fox example, stabilizers or polymerization inhibitors can be included in order to ensure an acceptable shelf life of cyanoacrylate adhesives. Polymerization accelerators can be incorporated into cyanoacrylate compositions for improving the curing speed of the adhesives, or in other words, additives for accelerating the polymerization reaction. The adverse effect of sterilization on cyanoacrylate compositions can be exacerbated, however, in the presence of polymerization accelerators. The invention provides for a desired method for sterilizing and storing cyanoacrylate compositions in the absence or presence of a polymerization accelerator.

The cyanoacrylate monomer compositions may contain one or more stabilizers or inhibitors including free radical stabilizers, anionic stabilizers, acidic stabilizers, mixtures thereof, and other suitable stabilizers, which preferably are mixed together with the cyanoacrylate monomers. The cyanoacrylate compositions may contain one or more free radical stabilizers. Free radical stabilizers may include without limitation, hydroquinone; catechol; butylated hydroxyl anisole (BHA). hydroquinone monomethyl ether and hindered phenols, such as butylated hydroxyanisol; 4-ethoxyphenol; butylated hydroxytoluene (BHT, 2,6-di-tert-butyl butylphenol), 4-methoxyphenol (MP); 3-methoxyphenol; 2-tert-butyl-4-methoxyphenol; 2,2-methylene-bis-(4-methyl-6-tert-butylphenol); and mixtures thereof. BHA is preferred.

The free radical stabilizer, if present, may be used in an amount less than about 40,000 ppm, less than about 30,000 ppm, less than about 25,000 ppm, less than about 20,000 ppm, less than about 15,000 ppm, less than about 10,000 ppm, less than about 5000 ppm, less than about 1000 ppm, or less than about 500 ppm. For example, the amount of free radical stabilizer may range from about 200 ppm to about 30,000 ppm, about 1000 ppm to about 30,000 ppm, about 2000 ppm to about 25,000 ppm, about 3000 ppm to about 20,000 ppm, about 3000 ppm to about 15,000 ppm, or about 5000 ppm to about 10,000 ppm.

The free radical stabilizer may comprise less than about 1.5% by weight of the cyanoacrylate composition. The free radical stabilizer may comprise about 0.1% to about 1.5%, 0.2% to about 1.5%, about 0.2% to about 0.8%, about 0.3% to about 1.2%, about 0.4% to about 1.0%, about 0.4% to about 0.6%, about 0.2% to about 1.0%, about 0.2% to about 0.8%, about 0.3% to about 1.5%, about 0.3% to about 1.0%, about 0.4% to about 1.5%, about 0.5% to about 1.5%, about 0.5% to about 1.2%, about 0.5% to about 1.0%, by weight of the cyanoacrylate composition.

The cyanoacrylate compositions may contain one or more acidic inhibitors or stabilizers, which may be in addition to the free radical stabilizer. Such acidic inhibitors may include without limitation sulfur dioxide, nitrogen oxide, boron oxide, phosphoric acid, ortho, meta, or para-phosphoric acid, acetic acid, benzoic acid, cyanoacetic acid, tri-fluoroacetic acid, tribromoacetic acid, trichloroacetic acid, boron trifluoride, hydrogen fluoride, perchloric acid, hydrochloric acid, hydrobromic acid, sulfonic acid, fluorosulfonic acid, chlorosulfonic acid, sulfuric acid, toluenesulfonic acid, and mixtures thereof. Sulfur dioxide is preferred.

The acid stabilizer, if present, may be used, for example, in an amount of about 50 ppm or less, about 40 ppm or less, about 30 ppm or less, about 25 ppm or less, about 20 ppm or less, about 15 ppm or less, about 10 ppm or less, about 7 ppm or less, or about 5 ppm or less. For example, the acid stabilizer may be present in an amount of about 1 ppm to about 50 ppm, about 2 ppm to about 50 ppm, about 5 ppm to about 50 ppm, about 5 ppm to about 20 ppm, about 5 ppm to about 19 ppm, about 10 ppm to about 50 ppm, about 2 ppm to about 40 ppm, about 5 ppm to about 30 ppm, about 10 ppm to about 25 ppm, about 10 ppm to about 15 ppm, about 10 ppm to about 25 ppm, about 10 ppm to about 20 ppm, about 10 ppm to about 19 ppm, about 15 ppm to about 30 ppm, about 15 ppm to about 25 ppm, about 15 ppm to about 20 ppm, about 17 ppm to about 20 ppm, about 17 ppm to about 19 ppm, about 18 ppm to about 30 ppm, about 18 ppm to about 25 ppm, about 12 ppm to about 20 ppm, or about 13 ppm to about 19 ppm.

The acid stabilizer may comprise less than about 0.05% by weight of the cyanoacrylate composition. The free radical stabilizer may comprise about 0.001% to about 0.01%, 0.001% to about 0.005%, 0.001% to about 0.004%, about 0.001% to about 0.003%, about 0.001% to about 0.002%, about 0.0015% to about 0.002%, about 0.0012% to about 0.002%, about 0.0015% to about 0.0019%, about 0.0013% to about 0.0019%, or about 0.0012% to about 0.0018% by weight of the cyanoacrylate composition.

Compared to cyanoacrylate compositions generally known in the art, the cyanoacrylate compositions stored in the package body of the invention contain much smaller amounts of acid stabilizer, such as sulfur dioxide, if present at all. For example, U.S. Pat. No. 5,480,935 provides for cyanoacrylate adhesive compositions with high amounts of sulfur dioxide as the anionic stabilizer, for example, on the order of about 150 to 250 ppm. U.S. Pat. Nos. 5,730,994 and 5,807,563 provide for about 50 to 500 ppm sulfur dioxide as the anionic polymerization inhibitor. In the examples of U.S. Publ. No. 2006/0062687, 100 ppm of sulfur dioxide was used to stabilize cyanoacrylate compositions.

Large amounts of stabilizer can increase the toxicity of the cyanoacrylate compositions. The cyanoacrylate compositions with small amounts of stabilizer packaged as described in the invention provide for long term stability of the cyanoacrylate formulations. For example, the desirable barrier property of the package system may work in concert to provide the stability of the cyanoacrylate compositions in the presence of such small amounts of stabilizer. It is thus an advantage of the invention to provide safe, stabilized cyanoacrylate compositions in the package systems.

The cyanoacrylate composition may include one or more polymerization accelerators, preferably mixed together with the cyanoacrylate monomers. Suitable polymerization accelerators may be selected from, without limitation, calixarenes and oxacalixarenes, silacrowns, crown-ethers, cyclodextrin and its derivatives, polyethers, aliphatic alcohol, various aliphatic carboxylic acid esters, benzoyl peroxide, amine compounds such as are triethyl amine, diethyl amine, butyl amine, isopropyl amine, tributyl amine, N,N,-dimethyl aniline, N,N-diethyl aniline, N,N-dimethyl-p-toluidine, N,N-dimethyl-m-toluidine, N,N-dimethyl-o-toluidine, dimethyl benzyl amine, pyridine, picoline, vinyl pyridine, ethanolamine, propanolamine and ethylene diamine, quaternary ammonium salts such as alkyl ammonium salts, amide-bonded ammonium salts, ester-bonded ammonium salts, ether-bonded ammonium salts and alkylimidazolinium salts, cyclosulfur compounds and derivatives, polyalkylene oxides and derivatives, and mixtures thereof.

In a preferred embodiment, a crown ether is used as the polymerization accelerator. Examples of crown ethers include, but are not limited to, 15-crown-5,18-crown-6, dibenzo-18-crown-6, tribenzo-18-crown-6, dicyclohexyl-18-crown-6, benzo-15-crown5, dibenzo-24-crown-8, dibenzo-30-crown-10, asym-dibenzo-22-crown-6, dimethylsila-11crown-4, dimethylsila-14-crown-5, dimethylsila-17-crown-6, dibenzo-14-crown-4, dicyclohexyl24-crown-8, asym-dibenzo-22-crown-6, cyclohexyl-12-crown-4,1,2-decalyl-15-crown-5, 1,2naphtho-15-crown-5,3,4,5-naphthyl-16-crown-5,1,2-methyl-benzo-18-crown-6, 1,2-methylbenzo-5,6-methylbenzo-18-crown-6,1,2-t-butyl-18-crown-6,1,2-vinylbenzo-15-crown-5,1,2-vinylbenzo-18-crown-6, 1,2t-butyl-cyclohexyl-18-crown-6, and 1,2-benzo-1,4-benzo-5oxygen-20-crown-7. The crown ether is preferably mixed together with the cyanoacrylate monomer.

The polymerization accelerator may be present in an amount less than about 6000 ppm, less than about 5000 ppm, less than about 4000 ppm, less than about 3000 ppm, less than about 2000 ppm, less than about 1000 ppm, less than about 750 ppm, less than about 500 ppm, less than about 250 ppm, less than about 100 ppm, or less than about 50 ppm. The amount of polymerization accelerator may range, for example from about 10 ppm to about 6000 ppm, about 10 ppm to about 2000 ppm, about 10 ppm to about 1200 ppm, about 10 ppm to about 1100 ppm, about 10 ppm to about 1000 ppm, about 20 ppm to about 2000 ppm, about 20 ppm to about 1500 ppm, about 20 ppm to about 1000 ppm, about 30 ppm to about 4000 ppm, about 30 ppm to about 3000 ppm, about 30 ppm to about 2000 ppm, about 30 ppm to about 1200 ppm, about 30 ppm to about 1000 ppm, about 40 ppm to about 1500 ppm, about 40 ppm to about 1200 ppm, about 40 ppm to about 1100 ppm, about 40 ppm to about 1000 ppm, about 50 ppm to about 3000 ppm, about 50 ppm to about 2000 ppm, about 50 ppm to about 1500 ppm, about 50 ppm to about 1200 ppm, about 50 ppm to about 1100 ppm, about 50 ppm to about 1000 ppm, about 75 ppm to about 1500 ppm, about 75 ppm to about 1000 ppm, about 100 ppm to about 5000 ppm, about 100 ppm to about 4000 ppm, about 100 ppm to about 3000 ppm, about 100 ppm to about 2000 ppm, about 100 ppm to about 1500 ppm, about 100 ppm to about 1300 ppm, about 100 ppm to about 1200 ppm, about 100 ppm to about 1100 ppm, about 100 to about 1000 ppm, about 200 ppm to about 1000 ppm, about 200 ppm to about 500 ppm, about 200 ppm to about 350 ppm, about 250 ppm to about 1250 ppm, about 250 ppm to about 1100 ppm, about 300 ppm to about 2000 ppm, about 300 ppm to about 1200 ppm, about 300 ppm to about 1100 ppm, about 500 ppm to about 1200 ppm, about 500 ppm to about 1100 ppm, about 500 ppm to about 1000 ppm, or about 60 ppm to about 1200 ppm, of the adhesive composition. For example, the amount of accelerator may range from about 200 ppm to about 350 ppm of the adhesive composition.

With the presence of a polymerization accelerator, the setting time of the cyanoacrylate composition upon irradiation sterilization may be in the range of about 5 to about 120 seconds, preferably about 10 to 90 seconds, and more preferably about 10 to about 60 seconds.

The cyanoacrylate composition may include one or more plasticizers or plasticizing agents. The plasticizing agent preferably does not contain any moisture and should not adversely affect the stability of the cyanoacrylate compositions. Examples of suitable plasticizers include, but are not limited to, tributyl citrate (TBC), acetyl tributyl citrate (ATBC), dimethyl sebacate, diethylsebacate, triethyl phosphate, tri(2-ethyl-hexyl)phosphate, tri(p-cresyl)phosphate, diisodecyl adipate (DIDA), glyceryl triacetate, glyceryl tributyrate, dioctyl adipate (DICA), isopropyl myrisate, butyl sterate, lauric acid, trioctyl trimelliate, dioctyl glutatrate (DICG), and mixtures thereof. Tributyl citrate, diisodecyl adipate and acetyl tributyl citrate may be preferred. The plasticizer, if present, is in an amount based on weight % of the cyanoacrylate composition of 20% or less, 15% or less, 10% or less, 7.5% or less, 5% or less, 2.5% or less or 1% or less.

The cyanoacrylate composition may include one or more dyes or colorants. In particular, the dyes may include derivatives of anthracene and other complex structures. Examples of suitable dyes include, but are not limited to, 1-hydroxy-4-[4-methylphenylamino]-9,10 anthracenedione (D&C violet No. 2); 9-(o-carboxyphenyl)-6-hydroxy-2,4,5,7-tetraiodo-3H-xanthen-3-one-, disodium salt, monohydrate (FD&C Red No. 3); disodium salt of 6-hydroxy-5-[(4-sulfophenyl)axo]-2-naphthalene-sulfonic acid (FD&C Yellow No. 6,); and 2-(1,3-dihydro-3-oxo-5-sulfo-2H-indole-2-ylidine)-2,3-dihydro-3-oxo-1H-ind-ole-5 sulfonic acid disodium salt (FD&C Blue No. 2), and the like. If present, small amounts of the dye may be used. For example, the cyanoacrylate composition may include one or more dyes in an amount of 1000 ppm or less, 500 ppm or less, 250 ppm or less, 100 ppm or less, or 50 ppm or less. For example, the dye may be present in an amount of about 1 ppm to about 1000 ppm, about 5 ppm to about 500 ppm, about 5 ppm to about 250 ppm, about 5 ppm to about 100 ppm, about 10 ppm to about 500 ppm, about 50 ppm to about 250 ppm, or about 75 ppm to about 100 ppm.

The cyanoacrylate composition may include one or more thickeners or thickening agents. Suitable thickening agents may include, but are not limited to, polycyanoacrylate, partial polymer of cyanoacrylate, polycaprolactone, copolymers of alkylacrylate and vinyl acetate, polyalkyl methacrylates, polyalkyl acrylates, lactic-glycolic acid copolymers, lactic acid-caprolactone copolymers, polyorthoesters, copolymers of alkyl methacrylates and butadiene, polyoxalates, triblock copolymers of polyoxypropylene flanked by two hydrophilic chains of polyoxyethylene, and mixtures thereof. Preferred thickening agents, if present, can be a partial polymer of cyanoacrylate as described in U.S. Pat. No. 8,198,344. Preferred thickening agents can also be triblock copolymers of polyoxyalkylene as described in U.S. Pat. No. 8,293,838. Preferably the thickening agent is miscible in the cyanoacrylate monomer compositions at room temperature. Biocompatible thickening agents are preferred for use in the medical field.

The amount of thickening agent, if present, may be present in an amount less than about 8000 ppm, less than about 7000 ppm, less than about 6000 ppm, less than about 5000 ppm. The amount of thickening agent may range, for example from about 10 ppm to about 8000 ppm, about 40 ppm to about 8000 ppm, about 60 ppm to about 7000 ppm, or about 100 ppm to about 6000 of the liquid adhesive composition.

### Sealed Container or Ampoule and First Sterilization

The primary package or ampoule contains the stable cyanoacrylate composition or adhesive. In other words, the ampoule has a chamber, and this chamber is filled with the stabilized cyanoacrylate monomer composition. The chamber preferably includes an opening through which the composition may be inserted in order to fill the chamber. The opening of the chamber is then closed by sealing it with a foil seal. The foil seal may be frangible.

To affix the foil to the sidewalls of the ampoule and cover the opening to the chamber, heat or adhesive may be applied. To provide a leak-free seal, certain temperatures and pressures may be applied. For example, the temperature used to connect the foil to the ampoule may be in the range of about 100° C. to about 200° C., preferably about 100° C. to about 180° C., and more preferably about 110° C. to about 170° C. The pressure used to connect the foil to the ampoule may be in the range of about 1 bar to about 50 bar, preferably about 1 bar to about 40 bar, and more preferably about 1 bar to about 15 bar. If the sealing temperature and/or pressure are too low, the seal between the ampoule and the foil seal may not be sufficiently tight to provide a leak-free container for storing and sterilizing cyanoacrylate adhesive. On the other hand, if the sealing temperature and/or pressure are too high, the ampoule may be deformed or the seal foil can be damaged during the assembly process.

The primary package or ampoule preferably is fabricated from gas—(e.g., oxygen) and/or moisture—(e.g., water vapor) impermeable materials. Preferably, the ampoule side walls are made of material with excellent gas and moisture barrier properties. The materials that may be used to construct the primary package, plastic container, or ampoule include without limitation, poly(ethylene-co-alkyl acrylates), acrylonitrile polymers and copolymers, polyamides, polyolefins, ethylene vinyl alcohol copolymers, ethylene-vinyl acetate copolymer, ethylene-alkyl acrylate-acrylic acid terpolymer, ethylene acrylic acid copolymer and polychlortrifluoethylene. In some aspects, the ampoule sidewalls may have a thickness between about 400 μm to about 5000 μm, about 400 μm to about 4000 μm, about 400 μm to about 3000 μm. about 500 μm to about 3000 μm, about 750 μm to about 2000 μm, or about 1000 μm to about 1500 μm. Preferably, the thickness of the ampoule sidewalls is at least about 400 μm.

Examples of poly(ethylene-co-alkyl acrylates) may include, for example, poly(ethylene-co-methyl acrylates) or poly(ethylene-co-ethyl acrylates). One preferred poly(ethylene-co-alkylacrylate) is poly(ethylene-co-butyl acrylates). Examples of acrylonitrile polymers and copolymers are polyacrylonitrile, acrylonitrile-methyl acrylate copolymer, acrylonitrile-acrylonitrile-octene copolymer, acrylonitrile-nonene copolymer, styrene-acrylonitrile copolymer, hexene copolymer, acrylonitrile-heptene copolymer, acrylonitrile-butadiene-styrene copolymer, acrylonitrile butadiene styrene copolymers, acrylonitrile-butyl acrylate copolymer, butadiene-acrylonitrile copolymer, acrylonitrile-octadecene copolymer, acrylonitrile-tridecene copolymer, and acrylonitrile-tetradecene copolymer. One preferred acrylonitrile copolymer is acrylonitrile-methyl acrylate copolymer. Examples of polyamides are polycaprolactam, poly(hexamethylene adipamide), polyphthalamide, m-phenylenediamine-isophthaloyl chrolide copolymer, and 1,4-phenylenediamine-terephthaloyl chloride copolymer. One preferred polyamide is poly(hexamethylene adipamide).

The foil seal may comprise a plurality of layers, including an inner layer and one or more outer layers. The multi-layer foil seal may include one or more inner layers; zero, one, two, three, four, five, six, seven, or more middle layers; and one, two, three, four, five, six, seven, or more outer layers. The inner layer may be made of a material with gas and moisture barrier properties, which includes without limitation, poly(ethylene-co-alkyl acrylates), acrylonitrile polymers and copolymers, polyamides, polyolefins, ethylene vinyl alcohol copolymers, ethylene-vinyl acetate copolymer, ethylene-alkyl acrylate-acrylic acid terpolymer, ethylene acrylic acid copolymer and polychlortrifluoethylene. The inner layer of the seal foil may have a thickness of about 10 μm to about 100 μm, preferably about 10 μm to about 90 μm and more preferably about 10 μm to about 80 μm. The inner layer of the seal foil may have a thickness of about 20 μm to about 80 μm, about 20 μm to about 90 μm, about 20 μm to about 70 μm, about 20 μm to about 60 μm, about 30 μm to about 100 μm, about 30 μm to about 80 μm, about 40 μm to about 80 μm, about 50 μm to about 80 μm, about 50 μm to about 100 μm, about 50 μm to about 90 μm, or about 60 μm to about 80 μm.

The outer layer and middle layer(s) of the seal foil may include, but are not limited to, polytetrafluoroethylene (PFTE); polyethylene terephthalate (PET); aluminum foil; polyethylene (PE) such as cross-linked high-density polyethylene (XLPE), linear low-density polyethylene (LLDPE), ultra low-density polyethylene, and very low-density polyethylene; polystyrene (PS), medium density polyethylene (MDPE), and high density polyethylene (HDPE); ethylene acrylic acid copolymer (EAA), polycarbonate (PC); polypropylene (PP); polystyrene (PS); polyvinylchloride (PVC); polybutylene terephthalate; polycarbonate (PC); polymethylpentene (PMP); polyketone (PK); naphthalate; polybutylene terephthalate; thermoplastic elastomer (TPE); mixtures thereof; and the like. The number of middle layers may range from 0 to about 5, preferably in the range of 0 to about 4, more preferably in the range of about 1 to about 3, and most preferably 1 or 2.

The middle layer and the outer layer of the seal foil may each independently have a thickness between about 5 μm to about 200 μm, preferably about 5 μm to 150 μm, and more preferably about 5 μm to about 100 μm. The middle and outer layers of the seal foil may independently have a thickness of about 5 μm to about 90 μm, 5 μm to about 80 μm, 5 μm to about 70 μm, about 5 μm to about 60 μm, about 10 μm to about 100 μm, about 10 μm to about 80 μm, about 10 μm to about 60 μm, about 10 μm to about 40 μm, about 20 μm to about 100 μm, about 20 μm to about 80 μm, about 20 μm to about 60 μm, about 20 μm to about 50 μm, about 30 μm to about 100 μm, about 30 μm to about 80 μm, or about 30 μm to about 70 μm. Although certain preferred thicknesses are described, those skilled in the art will appreciate that the thickness may be chosen so as to provide a consumer durable item that maintains structural integrity while also permitting some degree of package body flexing so as to permit dispensing of the contents.

Examples of multilayer seal foil layers suitable for use in this invention include without limitation, from innermost layer to outermost layer: acrylonitrile-methyl acrylate copolymer/ethylene acrylic acid copolymer/aluminum; acrylonitrile-methyl acrylate copolymer/aluminum/polypropylene; acrylonitrile-methyl acrylate copolymer/aluminum; acrylonitrile-methyl acrylate copolymer/polypropylene/aluminum; acrylonitrile-methyl acrylate/polyethylene/aluminum; acrylonitrile-methyl acrylate copolymer/ethylene acrylic acid copolymer/aluminum; acrylonitrile-methyl acrylate copolymer/aluminum/ethylene acrylic acid copolymer; acrylonitrile-methyl acrylate copolymer/aluminum/polyethylene; acrylonitrile-methyl acrylate copolymer/aluminum/polyethylene terephthalate; acrylonitrile-methyl acrylate copolymer/aluminum/polystyrene; ethylene-vinyl acetate copolymer/aluminum/polypropylene; ethylene-vinyl acetate copolymer/aluminum; ethylene-vinyl acetate copolymer/aluminum/polyethylene; ethylene-vinyl acetate copolymer/aluminum/polyethylene terephthalate; ethylene-vinyl acetate copolymer/aluminum/polystyrene; acrylonitrile-methyl acrylate copolymer/aluminum/polyethylene/polyethylene terephthalate; acrylonitrile-methyl acrylate copolymer/aluminum/polypropylene/polyethylene terephthalate; ethylene-alkyl acrylate-acrylic acid terpolymer/aluminum/polypropylene; ethylene-alkyl acrylate-acrylic acid terpolymer/aluminum; ethylene-alkyl acrylate-acrylic acid terpolymer/aluminum/polyethylene; ethylene-alkyl acrylate-acrylic acid terpolymer/aluminum/polyethylene terephthalate; ethylene-alkyl acrylate-acrylic acid terpolymer/aluminum/polystyrene, ethylene-vinyl acetate copolymer/polyethylene/polypropylene; ethylene-vinyl acetate copolymer/polystyrene/polyethylene; acrylonitrile-methyl acrylate copolymer/polypropylene/polyethylene; acrylonitrile-methyl acrylate copolymer/polyethylene/polypropylene; acrylonitrile-methyl acrylate copolymer/polysterene/polyethylene; acrylonitrile-methyl acrylate copolymer/polysterene/polypropylene; ethylene-vinyl acetate copolymer/polyvinylchloride/polypropylene; ethylene-vinyl acetate copolymer/polyvinylchloride/polyethylene; acrylonitrile-methyl acrylate copolymer/polyvinylchloride/polyethylene; polyacrylonitrile/polyvinylchloride/polypropylene; ethylene-alkyl acrylate-acrylic acid terpolymer/polypropylene/polyethylene; ethylene-alkyl acrylate-acrylic acid terpolymer/polyethylene/polypropylene; ethylene-alkyl acrylate-acrylic acid terpolymer/polystyrene/polyethylene; and ethylene-alkyl acrylate-acrylic acid terpolymer/polystyrene/polyethylene.

In some aspects, the foil seal is frangible. In some aspects, the connection between the foil seal and the ampoule sidewalls is frangible. Thus, upon compromising the seal, the cyanoacrylate composition housed within the chamber may flow out from the chamber, for example, for purposes of dispending the adhesive onto a surface to be adhered. In some aspects, the sidewalls of the ampoule are sufficiently flexible such that upon sufficient pressure, for example, by squeezing the sidewalls of the ampoule between the fingers of a user, the pressure within the ampoule compromises the foil seal or the connection between the foil seal and ampoule sidewalls such that the monomer composition may be released from the chamber. In some aspects, the sidewalls of the ampoule are hard but the ampoule seal is frangible. The ampoule can be connected to the applicator tip through a sleeve. One or more cutting members on the sleeve can break the seal. The cyanoacrylate composition in the ampoule may flow through the passageway in the sleeve onto the applicator tip. Flow of the adhesive composition can be controlled by squeezing the sidewalls of the sleeve.

The primary package (e.g., ampoule) may be of any suitable size, shape, design, or configuration known in the art. For example, the chamber of primary package/ampoule may have a volume up to about 20 mL. More specifically, the primary package may have a volume of about 0.1 mL to 20 mL, about 0.1 mL to 10 mL, about 0.2 mL to 6 mL, or about 0.3 mL to 5 mL. The primary package may be constructed as bottles, applicators, vials, ampoules, and the like. The package body may include, for example, a liquid containing area and a feed channel in fluid communication with the liquid containing area. Although an ampoule is exemplified herein, the ampoule may be replaced with any other suitable primary package design.

The primary container or ampoule containing the cyanoacrylate monomer composition is sterilized. Preferably, the primary container or ampoule containing the cyanoacrylate composition is sterilized by an irradiation method. In particular, the cyanoacrylate adhesive compositions in the primary package may be sterilized by Gamma, X-ray, Microwave, E-beam sterilization, or a combination thereof. Although these sterilization methods are described in detail herein, the sterilization may also comprise some combination of each of these irradiation techniques. The primary package is compatible with various irradiation methods for storing and sterilizing cyanoacrylate adhesive compositions. The package materials are stable under the desired dosage of the irradiation sterilization, and do not degrade as a result of the exposure to the radiation. The primary package provides a desired barrier to moisture so that it is compatible with the cyanoacrylate monomer compositions.

In one embodiment, the primary container and cyanoacrylate composition contained therein is sterilized with E-beam irradiation. The dose of E-beam irradiation applied to the package containing cyanoacrylate compositions should be sufficient enough to sterilize both the package and the adhesive inside. The E-beam irradiation can be in a suitable dosage amount, for example, of from about 5 to 50 kGy, and more preferably from about 12 to 25 kGy. E-beam irradiation may be conducted at any suitable temperature and pressure known in the art. Preferably, the E-beam irradiation may be conducted at ambient atmosphere conditions and the exposure time to the irradiation may be within 60 seconds, for example.

The absorbed dosage is specific to the type of the product and its density, the beam power, beam energy, scan height, and the speed at which the products moves through the electron beam. The power source for the electrons of E-beam irradiation is the linear accelerator, which is measured in kilo watts (KW). The larger the beam power is, the more product volume can be processed. The cyanoacrylate adhesive compositions stored in the primary packages may be irradiated at a beam power ranging from about 2 KW to about 30 KW, preferably about 5 KW to about 20 KW, and more preferably about 10 KW to about 20 KW.

E-beam irradiation for the cyanoacrylate compositions stored in the primary package involves the use of high-energy electrons. The beam energy may range from 1 million to 10 million electron volts (MeV), preferably 3 MeV to 10 MeV, and more preferably 5 to 10 MeV. The elevated energy levels are required to penetrate cyanoacrylate adhesive compositions, which are sterilized in the primary package or ampoule.

The processing speed also affects the delivered dosage of E-beam to the cyanoacrylate compositions stored in the primary packages. The processing speed may be controlled by the process conveyer that conveys the product through the beam at a given speed. The processing speed may range from about 1 to 20 feet per minute (fpm), preferably from about 2 to 15 fpm, and more preferably from about 4 to 10 fpm. The scan height of the E-beam may be in the range of about 16 inches to 30 inches, preferably in the range of about 20 to 30 inches, and more preferably in the range of about 25 to 30 inches.

In another embodiment, the primary container and cyanoacrylate composition contained therein is sterilized with gamma irradiation. The dose of gamma irradiation applied to the package containing cyanoacrylate compositions should be sufficient enough to sterilize both the package and the adhesive inside. The dose of gamma irradiation may range, for example, from about 5 to about 25 kGy, about 5 to about 20 kGy, about 5 to about 15 kGy, or about 5 to about 10 kGy. Standard Cobalt Co-60 may be used as the gamma ray source in sterilizing the compositions and packages of the invention.

In another embodiment, the primary container and cyanoacrylate composition contained therein is sterilized with X-ray irradiation. The dose of X-ray irradiation applied to the package containing cyanoacrylate compositions should also be sufficient enough to sterilize both the package and the adhesive inside. The dose of X-ray irradiation to cyanoacrylate compositions contained in the packages may range, for example, from about 5 kGy to about 40 kGy, about 5 kGy to about 30 kGy, about 5 kGy to about 25 kGy, or about 5 kGy to about 20 kGy. High energy electrons are preferably used for the X-ray sterilization of the liquid adhesive compositions. X-rays are generated as high-frequency and short-wavelength electromagnetic photons. Conventional X-ray technology may be suitable. The X-ray energy used to sterilize the primary container and the cyanoacrylate composition may range from about 1 million to about 10 million electron volts (MeV), about 3 MeV to 10 MeV, or about 3 to 7.5 MeV.

The cyanoacrylate-based adhesive compositions contained within the primary package can be sterilized via Gamma, X-ray, and/or E-beam irradiations in a box containing a large amount of containers provided in the box. For example, the box can include a large amount of applicators. The box may include 5,000 applicators or less, 4,000 applicators or less, 3,000 applicators or less, 2,500 applicators or less, 2,000 applicators or less, 1,500 applicators or less, or 1,000 applicators or less. More specifically, the box may contain from about 200 to about 4,000, about 200 to about 3,000, or about 200 to 2,500 applicators containing the cyanoacrylate adhesive compositions, for example. The density of the box containing a large amount of applicators can range from about 0.04 to about 0.4 g/cm³, preferably from about 0.05 to about 0.4 g/cm³, and more preferably from about 0.05 to about 0.3 g/cm³.

Before or after the irradiation sterilization, the primary container or ampoule containing the cyanoacrylate composition may be further assembled into an applicator body. For example, the applicator body may have an attached applicator tip for dispensing the cyanoacrylate adhesive composition. The applicator may include a reservoir container and a sponge application tip, for example. The sponge tip may be saturated with liquid adhesive once it is folded over so that adhesive can be dispensed uniformly onto the wound site. The container size and sponge tip can be varied dependent on the volume of the adhesive. The applicator may have a volume of about 0.1 mL to 10 mL, preferably about 0.2 mL to 6 mL, and more preferably about 0.3 mL to 5 mL. In order to inhibit premature polymerization, the volume of the applicator is preferably about 50 to 80 percent and more preferably 60 to 80 percent filled by the cyanoacrylate adhesives.

The cyanoacrylate adhesive compositions in the packages such as, for example, an applicator with an overpack can be sterilized by E-beam, Gamma, or X-ray irradiation in different configurations. Such packages may contain adhesive compositions in an amount of, for example, from about 0.1 mL to about 10 mL, preferably from about 0.1 mL to about 5 mL, and more preferably from about 0.2 mL to about 5 mL.

With respect to the viscosity of the cyanoacrylate composition, it will be appreciated by those skilled in the art that the viscosity of cyanoacrylate adhesive compositions generally increases following irradiation. It is preferred, however, that in accordance with the storage container of the invention, the viscosity does not change dramatically, either higher or lower, during or subsequent to the irradiation process.

The viscosity of the cyanoacrylate monomer compositions including the thickening agents stored in the primary package may change upon irradiation sterilization. The change of the viscosity may depend, for example, on the presence or absence of certain additives in the composition, including a thickening agent (e.g., a partial polymer of cyanoacrylate may be used as the thickening agent to prepare the cyanoacrylate compositions with a desired level of high viscosity) and/or a polymerization accelerator.

After irradiation sterilization in the primary package, the viscosity of the cyanoacrylate composition may change, including an increase or decrease to a second viscosity. The change in viscosity of the cyanoacrylate adhesive compositions, after the sterilization, may vary, for example, depending on the original or first viscosity and the presence of additives such as a polymerization accelerator or thickening agent. When stored in the primary package, however, the change in viscosity is preferably minimal. Preferably, the second viscosity or viscosity of the composition after sterilization (in the primary package/ampoule) is within about 1% to about 100% of the initial or first viscosity of the composition, before sterilization. In some embodiments, the viscosity of the composition after sterilization is within about 5% to about 300% of the initial viscosity of the composition, before sterilization. The viscosity may change about 5% to about 10%, about 5% to about 15%, about 5% to about 20%, about 7% to about 10%, about 7% to about 15%, about 8% to about 12%, about 8% to about 15%, about 8% to about 20%, about 10% to about 100%, about 10% to about 80%, about 10% to about 60%, about 10% to about 40%, about 10% to about 30%, about 10% to about 20%, about 20% to about 100%, about 20% to about 60%, about 20% to about 50%, about 20% to about 40%, about 20% to about 30%, about 30% to about 300%, about 30% to about 200%, about 30% to about 150%, about 30% to about 100%, about 30% to about 50%, about 40% to about 300%, about 40% to about 200%, about 40% to about 150%, about 40% to about 100%, about 40% to about 80%, about 40% to about 80%, about 50% to about 300%, about 50% to about 200%, about 50% to about 150%, about 50% to about 100%, about 50% to about 90%, about 50% to about 80%, about 60% to about 200%, about 60% to about 100%, about 70% to about 200%, about 70% to about 100%, about 80% to about 100% of the initial viscosity.

This second viscosity, the viscosity of the composition in the ampoule after irradiation sterilization, but before the second sterilization step, may be less than about 400 cPs, less than about 300 cPs, less than about 200 cPs, less than about 100 cPs, less than about 50 cPs, less than about 25 cPs, less than about 20 cPs, less than about 15 cPs, less than about 10 cPs, or less than about 7 cPs. In particular, the second viscosity of the cyanoacrylate composition may be in the range of about 3 cPs to about 100 cPs, about 3 cPs to about 50 cPs, about 3 cPs to about 20 cPs, about 3 cPs to about 10 cPs, about 4 cPs to about 15 cPs, about 5 cPs to about 10 cPs, about 5 cPs to about 7 cPs, about 5 cPs to about 9 cPs, about 5 cPs to about 8 cPs, about 5 cPs to about 100 cPs, about 5 cPs to about 50 cPs, about 5 cPs to about 20 cPs, about 5 cPs to about 15 cPs, about 10 cPs to about 20 cPs, about 10 cPs to about 25 cPs, about 6 cPs to about 7 cPs, about 6 cPs to about 8 cPs, about 6 cPs to about 10 cPs, about 6 cPs to about 14 cPs, about 7 cPs to about 12 cPs, about 7 cPs to about 10 cPs, about 10 cPs to about 60 cPs, about 10 cPs to about 15 cPs, about 15 cPs to about 20 cPs, about 15 cPs to about 25 cPs, about 15 cPs to about 30 cPs, about 10 cPs to about 15 cPs, about 10 cPs to about 20 cPs, about 20 cPs to about 25 cPs, about 20 cPs to about 30 cPs, about 25 cPs to about 50 cPs, about 25 cPs to about 75 cPs, or about 25 cPs to about 30 cPs, prior to the second sterilization.

Generally speaking, after the cyanoacrylate composition contained within the primary package or ampoule is sterilized by irradiation, the viscosity of the cyanoacrylate formulation is substantially the same as the initial viscosity (pre-irradiation). In particular, the primary package or ampoule is sterilized by irradiation while maintaining the viscosity of the stable cyanoacrylate composition such that a change in the viscosity (i.e., from the first viscosity to the second viscosity) is no more than a small amount. In some aspects, the change between the initial viscosity of the cyanoacrylate composition and the second viscosity of the cyanoacrylate composition after irradiation sterilization is preferably less than about 30 cPs, less than about 25 cPs, less than about 22 cPs, less than about 20 cPs, less than about 19 cPs, less than about 18 cPs, less than about 17 cPs, less than about 16 cPs, less than about 15 cPs, less than about 14 cPs, less than about 13 cPs, less than about 12 cPs, less than about 11 cPs, less than about 10 cPs, less than about 9 cPs, less than about 8 cPs, less than about 7 cPs, less than about 6 cPs, less than about 5 cPs, less than about 4 cPs, less than about 3 cPs, less than about 2 cPs, or less than about 1 cPs.

The invention provides for a suitable package system for cyanoacrylate compositions, which can be sterilized via irradiation sterilization while maintaining the viscosity of the cyanoacrylate composition. The primary package is made of materials which are gas/moisture resistant. The barrier property and stability upon irradiation of the primary package make it a suitable container to sterilize and store the cyanoacrylate compositions. The cyanoacrylate compositions, even in the presence of small amounts of stabilizers, can be packaged in the primary package and can be sterilized in the packaging with irradiation sterilization.

### Secondary Overpack and Second Sterilization

The secondary overpack houses or contains the primary package or ampoule already containing the stable and sterilized cyanoacrylate composition. In other words, the package body of the primary package is wrapped with or otherwise surrounded by a protective secondary overpack.

The secondary overpack may include a front wrapper and a back wrapper. The material for the front wrapper may include, but are not limited to, polyethylene (PE) polytetrafluoroethylene (PFTE); polyethylene terephthalate (PET); amorphous polyethylene terephthalate (APET), polystyrene (PS), polycarbonate (PC); polypropylene (PP); polystyrene (PS); polyvinylchloride (PVC); and thermoplastic elastomer (TPE); mixtures thereof. The front wrapper may be of any suitable thickness. For example, the front wrapper may have a thickness of between about 100 μm to about 1000 μm, preferably about 200 μm to about 800 μm, and more preferably about 300 μm to about 600 μm.

A portion or the entire secondary overpack may be made of one or more materials that is gas permeable. The front wrapper, the back wrapper, or both the front and back wrappers of the secondary overpack preferably are gas permeable such that the secondary overpack is suitable for use with a gaseous sterilization, such as ethylene oxide (ETO) sterilization, for example. The material used for the back wrapper of the secondary overpack may, include without limitation, ultra low density of polyethylene, a medical grade Kraft paper coated with a low density polyethylene, low density nylon, cellophanelpolyethylene laminate, phenoxy, mylarlpolyethylene laminate, and the like. The back wrapper may have any suitable thickness. For example, the back wrapper may have a thickness of between about 20 μm to about 200 μm, preferably about 30 μm to about 150 μm, and more preferably about 50 μm to about 100 μm. The back wrapper of the secondary packaging may preferably include a medical grade paper coated with heat sealant.

The front wrapper and back wrapper are preferably joined together after they are positioned to surround the ampoule. The front wrapper and the back wrapper of the secondary overpack may be heat-sealed together, for example, under elevated temperature and pressure. The front and back wrapper may each comprise a portion of a blister such that when brought together, they form a complete blister, and in this blister may be contained the primary container or ampoule. The temperature used to seal the front wrapper and back wrapper of the overpack together may be selected by one of ordinary skill in the art. For example, the heat sealing temperature may be in the range of about 110° C. to about 250° C., preferably about 110° C. to about 200° C., and more preferably about 120° C. to about 180° C. The pressure used to seal the front wrapper and the back wrapper may be in the range of about 1 NM (Newton Meter) to about 40 NM, preferably about 1 NM to about 30 NM, and more preferably about 1 NM to about 20 NM.

Once the adhesive container is assembled into the sleeve with the applicator tip, the primary package (containing the monomer composition) together with the secondary overpack may be further sterilized by chemical sterilization. The chemical sterilization method may include without limitation, ozone sterilization, ethylene oxide sterilization, hydrogen peroxide sterilization, formaldehyde sterilization, and peracetic acid sterilization. In an exemplary embodiment, the chemical sterilization for sterilizing the primary package containing the cyanoacrylate composition and the secondary overpack is ethylene oxide (ETO) sterilization.

When the cyanoacrylate composition contained within the primary package and also contained within the secondary overpack is sterilized a second time, the second time by chemical sterilization, the viscosity of the cyanoacrylate composition is maintained at or approximate to the viscosity of the initial viscosity and/or the second viscosity. Nevertheless, the viscosity may change following the chemical sterilization. The secondary overpack may be sterilized by chemical sterilization while substantially maintaining the second viscosity of the stable cyanoacrylate composition such that a change in the viscosity is no more than a small amount. After the second sterilization, the cyanoacrylate monomer composition has a third viscosity. Preferably, the change in viscosity from the second to the third viscosity is less than about 30 cPs, less than about 25 cPs, less than about 22 cPs, less than about 20 cPs, less than about 18 cPs, less than about 17 cPs, less than about 16 cPs, less than about 15 cPs, less than about 14 cPs, less than about 13 cPs, less than about 12 cPs, less than about 11 cPs, less than about 10 cPs, less than about 9 cPs, less than about 8 cPs, less than about 7 cPs, less than about 6 cPs, less than about 5 cPs, less than about 4 cPs, less than about 3 cPs, less than about 2 cPs, or less than about 1 cPs.

The viscosity may change from the second to the third viscosity may be about 5% to about 10%, about 5% to about 20%, about 7% to about 10%, about 8% to about 20%, about 8% to about 15%, about 10% to about 100%, about 10% to about 80%, about 10% to about 60%, about 10% to about 40%, about 10% to about 30%, about 10% to about 20%, about 20% to about 100%, about 20% to about 60%, about 20% to about 50%, about 20% to about 40%, about 20% to about 30%, about 30% to about 300%, about 30% to about 200%, about 30% to about 150%, about 30% to about 100%, about 30% to about 50%, about 30% to about 40%, about 40% to about 300%, about 40% to about 200%, about 40% to about 150%, about 40% to about 100%, about 40% to about 80%, about 40% to about 80%, about 40% to about 50%, about 50% to about 300%, about 50% to about 200%, about 50% to about 150%, about 50% to about 100%, about 50% to about 90%, about 50% to about 80%, about 60% to about 200%, about 60% to about 100%, about 70% to about 200%, about 70% to about 100%, about 80% to about 100% of the second viscosity.

This third viscosity, the viscosity of the composition in the ampoule housed in the overpack after irradiation sterilization and chemical sterilization but before simulated aging conditions (80 degrees C. for about 12 or about 13 days), may be less than about 400 cPs, less than about 300 cPs, less than about 200 cPs, less than about 100 cPs, less than about 50 cPs, less than about 25 cPs, less than about 20 cPs, less than about 15 cPs, less than about 10 cPs, or less than about 7 cPs. In particular, the third viscosity of the cyanoacrylate composition may be in the range of about 3 cPs to about 100 cPs, about 3 cPs to about 50 cPs, about 3 cPs to about 20 cPs, about 3 cPs to about 12 cPs, about 4 cPs to about 15 cPs, bout 4 cPs to about 12 cPs, about 5 cPs to about 10 cPs, about 5 cPs to about 7 cPs, about 5 cPs to about 9 cPs, about 5 cPs to about 8 cPs, about 5 cPs to about 100 cPs, about 5 cPs to about 50 cPs, about 5 cPs to about 20 cPs, about 5 cPs to about 15 cPs, about 10 cPs to about 15 cPs, about 10 cPs to about 20 cPs, about 10 cPs to about 25 cPs, about 6 cPs to about 7 cPs, about 6 cPs to about 10 cPs, about 6 cPs to about 14 cPs, about 7 cPs to about 12 cPs, about 7 cPs to about 10 cPs, about 10 cPs to about 60 cPs, about 10 cPs to about 25 cPs, about 15 cPs to about 20 cPs, about 15 cPs to about 25 cPs, about 15 cPs to about 30 cPs, about 10 cPs to about 15 cPs, about 10 cPs to about 20 cPs, about 20 cPs to about 25 cPs, about 20 cPs to about 30 cPs, about 25 cPs to about 50 cPs, about 25 cPs to about 75 cPs, or about 25 cPs to about 30 cPs, prior to the advanced aging storage.

The invention provides for a suitable package system for cyanoacrylate compositions, which can be sterilized twice: first via irradiation sterilization and second via chemical sterilization, while substantially maintaining the initial viscosity of the cyanoacrylate composition throughout the entire process. The cyanoacrylate compositions, even in the presence of thickening agents and/or polymerization accelerators, can be packaged in the primary package and subsequently in the secondary overpack and can be sterilized in the packaging with chemical sterilization. The combination of the primary package with the stable cyanoacrylate compositions and the two-step sterilization provide for an extended shelf life of the cyanoacrylate compositions of at least one year, preferably at least two years.

The twice-sterilized package system, including the ampoule containing a sterilized cyanoacrylate monomer composition and the overpack, maintains stability to the cyanoacrylate composition within the package system when stored over time, particularly at normal or ambient temperature and humidity conditions attendant to shelf storage of such a package system. The stability is reflected in minimal viscosity changes in the composition over the storage time. The sterilized composition is viscosity-stable for at least two years of shelf storage. Two years of shelf storage may be simulated with a standard advanced aging test, which subjects the package system (including the composition therein) to 80 degrees C. for at least 12 days, and in some aspects 13 days.

Following the accelerated aging test, the viscosity of the composition may change from the third viscosity to a fourth viscosity. Following the accelerated aging test, the composition substantially maintains the third viscosity such that a change in the viscosity is no more than a small amount. Preferably, the change in viscosity from the third viscosity to the fourth viscosity is less than about 400 cPs, less than about 300 cPs, less than about 200 cPs, less than about 150 cPs, less than about 100 cPs, less than about 75 cPs, less than about 50 cPs, less than about 25 cPs, less than about 22 cPs, less than about 20 cPs, less than about 18 cPs, less than about 17 cPs, less than about 16 cPs, less than about 15 cPs, less than about 14 cPs, less than about 13 cPs, less than about 12 cPs, less than about 11 cPs, less than about 10 cPs, less than about 9 cPs, less than about 8 cPs, less than about 7 cPs, less than about 6 cPs, less than about 5 cPs, less than about 4 cPs, less than about 3 cPs, less than about 2 cPs, or less than about 1 cPs.

The viscosity change from the third to the fourth viscosity may be about 5% to about 10%, about 5% to about 20%, about 7% to about 10%, about 10% to about 100%, about 10% to about 80%, about 10% to about 60%, about 10% to about 40%, about 10% to about 30%, about 10% to about 20%, about 20% to about 100%, about 20% to about 60%, about 20% to about 50%, about 20% to about 40%, about 20% to about 30%, about 30% to about 500%, about 30% to about 400%, about 30% to about 300%, about 30% to about 200%, about 30% to about 150%, about 30% to about 100%, about 30% to about 50%, about 30% to about 40%, about 40% to about 500%, about 40% to about 400%, about 40% to about 300%, about 40% to about 200%, about 40% to about 150%, about 40% to about 100%, about 40% to about 80%, about 40% to about 80%, about 40% to about 50%, about 50% to about 500%, about 50% to about 400%, about 50% to about 300%, about 50% to about 200%, about 50% to about 150%, about 50% to about 100%, about 50% to about 90%, about 50% to about 80%, about 60% to about 200%, about 60% to about 100%, about 70% to about 200%, about 70% to about 100%, about 80% to about 100%, about 100% to about 200%, about 100% to about 300%, about 100% to about 350%, about 200% to about 300%, or about 200% to about 400% of the third viscosity.

This fourth viscosity, the viscosity of the composition in the ampoule housed in the overpack after irradiation sterilization, chemical sterilization, and simulated advanced aging conditions (80 degrees C. for about 12 or about 13 days), may be less than about 400 cPs, less than about 300 cPs, less than about 200 cPs, less than about 100 cPs, less than about 50 cPs, less than about 25 cPs, less than about 20 cPs, less than about 15 cPs, less than about 10 cPs, or less than about 7 cPs. In particular, the third viscosity of the cyanoacrylate composition may be in the range of about 3 cPs to about 100 cPs, about 3 cPs to about 50 cPs, about 3 cPs to about 20 cPs, about 4 cPs to about 15 cPs, about 5 cPs to about 10 cPs, about 5 cPs to about 7 cPs, about 5 cPs to about 9 cPs, about 5 cPs to about 8 cPs, about 5 cPs to about 100 cPs, about 5 cPs to about 50 cPs, about 5 cPs to about 35 cPs, about 5 cPs to about 20 cPs, about 5 cPs to about 15 cPs, about 10 cPs to about 20 cPs, about 10 cPs to about 25 cPs, about 10 cPs to about 30 cPs, about 10 cPs to about 35 cPs, about 10 cPs to about 40 cPs, about 6 cPs to about 7 cPs, about 6 cPs to about 10 cPs, about 6 cPs to about 14 cPS, about 6 cPs to about 26 cPs, about 7 cPs to about 12 cPs, about 7 cPs to about 10 cPs, about 10 cPs to about 60 cPs, about 10 cPs to about 45 cPs, about 10 cPs to about 40 cPs, about 10 cPs to about 35 cPs, about 10 cPs to about 30 cPs, about 15 cPs to about 20 cPs, about 15 cPs to about 25 cPs, about 15 cPs to about 30 cPs, about 15 cPs to about 35 cPs, about 15 cPs to about 45 cPs, about 15 cPs to about 50 cPs, about 10 cPs to about 15 cPs, about 20 cPs to about 25 cPs, about 20 cPs to about 30 cPs, about 20 cPs to about 35 cPs, about 20 cPs to about 40 cPs, about 20 cPs to about 50 cPs, about 25 cPs to about 50 cPs, about 25 cPs to about 75 cPs, about 25 cPs to about 30 cPs, about 25 cPs to about 35 cPs, about 26 cPs to about 29 cPs, or about 26 cPs to about 30 cPs.

The fourth viscosity may be about 10% to about 500% higher than the first viscosity. The fourth viscosity may be about 5% to about 50%, about 5% to about 100%, about 10% to about 350%, about 10% to about 300%, about 10% to about 250%, about 10% to about 100%, about 30% to about 400%, about 30% to about 350%, about 30% to about 300%, about 30% to about 200%, about 30% to about 100%, about 50% to about 500%, about 50% to about 400%, about 50% to about 350%, about 50% to about 300%, about 50% to about 150%, about 70% to about 400%, about 70% to about 350%, about 70% to about 300%, about 70% to about 200%, about 100% to about 500%, about 100% to about 400%, about 100% to about 350%, about 100% to about 330%, about 100% to about 300%, about 100% to about 250%, about 150% to about 400%, about 150% to about 350%, about 150% to about 250%, about 150% to about 200%, about 200% to about 400%, about 200% to about 350%, about 200% to about 300%, about 250% to about 400%, about 250% to about 350%, about 300% to about 350%, or about 310% to about 340% of the first viscosity.

### Process of Producing the Package System

According to one embodiment, the method or process for producing and sterilizing the cyanoacrylate compositions in a package system may include: (a) inserting a stabilized cyanoacrylate composition having a desires viscosity into an ampoule such as any ampoule described or exemplified herein, (b) sealing the ampoule containing the stable cyanoacrylate composition with a foil seal, including any foil seal described or exemplified herein, (c) sterilizing the sealed ampoule containing the stabilized cyanoacrylate composition via irradiation sterilization, (d) placing the sterilized and sealed ampoule into an overpack to provide a package system, and (f) sterilizing the package system via a chemical sterilization. Optionally, the methods may include preparing a stable cyanoacrylate composition, which may optionally comprise one or more additives such as a polymerization accelerator, plasticizer, or thickener.

According to another embodiment, a method or process for producing and sterilizing the cyanoacrylate compositions in a package system may include: (a) preparing cyanoacrylate monomer with a purity of between about 97-99% by weight; (b) stabilizing the cyanoacrylate compositions with free radical and anionic polymerization inhibitors and dissolving a polymerization accelerator in the cyanoacrylate compositions; (c) filling the cyanoacrylate compositions into a primary package and sealing the primary package; (d) sterilizing the cyanoacrylate compositions in the primary package via one or more irradiation methods; (e) assembling the primary package into the applicator with the applicator tip and packing the assembled device into a secondary overpack; and (f) sterilizing the whole package system via a chemical sterilization.

This method or process provides a number of benefits, including, but not limited to: (1) allowing direct contact between the cyanoacrylate composition and an optional polymerization accelerator during the sterilization and storage; (2) superior shelf life stability; (3) inhibiting or preventing adverse side effects of irradiation on the packaging materials; and (4) producing a sterile and shelf-stable cyanoacrylate monomer composition.

The process provides for a way of sterilizing the cyanoacrylate compositions with a miscible polymerization accelerator, which is preferably mixed together with the cyanoacrylate composition during sterilization. Generally, polymerization accelerators may induce polymerization and/or make polymerization proceed rapidly such that the polymerization accelerator and the adhesive composition had to be separated during sterilization, and maintained separated until just prior to applying the composition to the materials to be adhered together in order to prevent the premature polymerization of the adhesive induced by the sterilization and facilitated by the accelerator. This is evidenced by U.S. Pat. Nos. 6,579,469, 6,620,846, and 5,928,611, as well as in U.S. Publ. Nos. 2005/0047846, 2007/0078207, 2010/0330027, 2010/0269749, and 2008/0241249.

Second, the stable cyanoacrylate composition along with the method of sterilizing the cyanoacrylate composition in the package system provides for sterile cyanoacrylate compositions with an extended shelf life of at least 12 months, preferably at least 15 months, more preferably at least 18 months, and even more preferably at least 24 months. Thus, the package system is capable of being stored at room temperature (e.g., about 20° C. to about 25° C.) for long periods of time without substantially increasing in viscosity, deteriorating, degrading, polymerizing, or otherwise reacting or changing in properties. The shelf life of a product may be evaluated by any suitable technique. For example, the package system may undergo an accelerated aging test at elevated temperature to evaluate the shelf life stability of the cyanoacrylate compositions. This test can be performed in an oven at 80° C. for a period of 13 days. Based on ASTM F1980-2, 13 days accelerated aging at 80° C. correlates to 2 years of shelf life at ambient temperatures. Similarly, a real time shelf life study could also be conducted. At the end of 2 years of shelf life evaluated by real time study or accelerated aging studies, cyanoacrylate compositions in the package system sterilized by irradiation method preferably have a viscosity of less than about 400 cPs, more preferably less than about 300 cPs, and most preferably less than about 200 cPs.

Third, the method of sterilizing cyanoacrylate compositions in the package system can offset the potential side effect of irradiation sterilization on other parts of the package system such as the applicator body that holds the primary container, the applicator tip for dispensing the adhesive, and the secondary overpack. It is known that irradiations have various effects on different package materials made of polymers, copolymers or other components, such as changing color, affecting tensile properties, and oxidizing of the package material upon irradiation. In particular, irradiation can turn most of polymer-based white packaging materials into yellow, which makes the package components such as the applicator body, the applicator tip and the secondary overpack cosmetically and aesthetically undesirable or unacceptable. The method of sterilizing cyanoacrylate composition disclosed herein only exposes the primary container for the adhesive composition to irradiation, while the rest components of the package system are sterilized by a chemical sterilization, which can effectively inhibit the side effect of irradiation on the packaging materials.

Fourth, a stable and sterile cyanoacrylate product may be produced. For example, a sterility assurance level (SAL) can be obtained at a minimum of 10⁻³, which means that the probability of a single unit being non-sterile after sterilization is 1 in 1000. In more preferred embodiments, the sterility assurance level may be at least 10⁻⁶. The sterility of the cyanoacrylate monomer composition packaged in the package system after E-beam sterilization was analyzed by Bacteriostasis and Fungistasis tests. After testing with challenging microorganisms, such as Bacillus subtilis, Candida albicans, and Aspergillus niger, no growth of the microorganisms was observed for the cyanoacrylate adhesive in the package system after E-beam sterilization, indicating the sterility of the cyanoacrylate adhesives.

The following non-limiting examples are intended to further illustrate, but not to limit, the invention.

### Example 1

### Shelf Life Stability

As summarized in Table 1, three formulations were considered under different irradiation techniques: (1) Formulation 1: stabilized 2-octyl cyanoacrylate mixed together with a polymerization accelerator; (2) Formulation 2: stabilized mixture of 2-octyl cyanoacrylate and n-butyl cyanoacrylate; and (3) Formulation 3: stabilized 2-octyl cyanoacrylate mixed together with a plasticizer. The formulations, respectively, were stored in a container made of acrylonitrile-methyl acrylate copolymer and multi-layer seal foil or multiple layer materials with acrylonitrile-methyl acrylate copolymer as the adhesive contacting inner layer. The ampoule container was initially sterilized by irradiation method, which may be assembled into the applicator with the applicator tip. The ampoule or the ampoule-applicator assembly was then packaged into the overpack, which was re-sterilized by a chemical sterilization. The compositions in the other comparative containers, such as polypropylene (PP), low density polyethylene (LDPE), etc. were sterilized by irradiation only.

As shown in Table 1, the cyanoacrylate compositions stored in the package system can provide a shelf life of at least 24 months under various irradiation techniques such as E-beam, Gamma, and X-ray. This was confirmed by a real time shelf life study and accelerated aging test by storing the sterilized package containing a sterilized cyanoacrylate composition at 80 degrees C. for 13 days. In comparison, however, the same cyanoacrylate adhesive compositions contained in other package systems (* denotes comparative examples) made of only low density polyethylene (LDPE), high density polyethylene (HDPE), and polypropylene, amber HDPE, glass, and polyethylene terephthalate glycol were not found to be as stable upon irradiation sterilization. The cyanoacrylate compositions packaged in other systems as listed below were cured in about a month after the irradiation sterilization, exhibiting an unacceptable shelf life.

The cyanoacrylate compositions packaged in the package system provide the extended shelf life of at least two years after irradiation sterilization. These observations demonstrate the uniqueness of the package system disclosed herein as a suitable container for the cyanoacrylate compositions.

TABLE 1
Shelf life stability of cyanoacrylate compositions in different
packages under various irradiation sterilizations

| | | | Shelf life stability of the |
|---|---|---|---|
| Composition | Package component | Irradiation | adhesive in the package |
| Formulation 1 | Package system of the | E-beam | At least 24 months |
| | invention | | |
| Formulation 2 | Package system of the | E-beam | At least 24 months |
| | invention | | |
| Formulation 1 | Package system of the | Gamma | At least 24 months |
| | invention | | |
| Formulation 1 | Package system of the | X-ray | At least 24 months |
| | invention | | |
| Formulation 3 | Package system of the | E-beam | At least 24 months |
| | invention | | |
| Formulation 2 | Polypropylene* | E-beam | Cured in about a month |
| Formulation 2 | Low density polyethylene* | E-beam | Cured in about a month |
| Formulation 1 | Amber HPDE* | E-beam | Cured in about a month |
| Formulation 1 | Polypropylene* | E-beam | Cured in about a month |
| Formulation 1 | Amber glass* | E-beam | Cured in about a month |
| Formulation 1 | Low density polyethylene* | E-beam | Cured in about a month |
| Formulation 2 | High density polyethylene* | E-beam | Cured in about a month |
| Formulation 3 | High density polyethylene* | E-beam | Cured in about a month |
| Formulation 1 | High density polyethylene* | E-beam | Cured in about a month |

(*denotes comparative examples)

It is thus an advantage of the invention to provide a packaging system made of multiple layers of different materials for storing and sterilizing cyanoacrylate adhesive compositions. The desired barrier properties of the packaging material make it compatible with various irradiations to provide sterile cyanoacrylate compositions with an extended shelf life of at least 2 years.

### Example 2

### Viscosity

The viscosity of the cyanoacrylate compositions were measured by a Brookfield DV-II+ viscometer at room temperature (25° C.) and atmospheric pressure. The spindle and cup were cleaned with acetone after each measurement. About 0.5 ml of the cyanoacrylate composition was put into the cup and the cup was brought into position and slowly secured with the retaining arm. The motor was turned on after the sample was equilibrated in the cup. The viscosity of the cyanoacrylate composition was measured in triplicate. Any residue was removed with acetone prior to next sample measurement.

The performance of the cyanoacrylate monomer compositions stored in the package system shows essentially no change or only slight changes in viscosity upon irradiation sterilization. Viscosity of the cyanoacrylate monomer compositions was measured before and after the irradiation sterilization. The sterilized cyanoacrylate monomer compositions show comparable viscosity to the same composition before irradiation sterilization. As an example, the viscosity of 2-octyl cyanoacrylate composition after E-beam sterilization is 3.47 cPs, compared to 4.90 cPs of the composition before E-beam sterilization. As shown in Table 2, the viscosity of the cyanoacrylate composition in the ampoule only shows essentially no change or only minimal change in viscosity after the irradiation sterilization compared to that prior to sterilization. The composition for Lots 1-3 included 2-octyl cyanoacrylate as the adhesive monomer, and underwent Gamma irradiation sterilization only.

TABLE 2
Viscosity of 3 lots of cyanoacrylate compositions included in the ampoule
before and after irradiation sterilization only at 11.6-14.0 kGy

| | Viscosity (cp) | | | | | | | |
|---|---|---|---|---|---|---|---|---|
| | Before Sterilization | | | | After sterilization | | | |
| Sample | 1 | 2 | 3 | Avg. | 1 | 2 | 3 | Avg. |
| Lot 1 | 6.05 | 6.03 | 6.03 | 6.04 | 6.77 | 6.74 | 6.72 | 6.74 |
| Lot 2 | 6.21 | 6.21 | 6.23 | 6.22 | 6.64 | 6.67 | 6.79 | 6.70 |
| Lot 3 | 6.41 | 6.39 | 6.39 | 6.40 | 6.82 | 6.85 | 6.90 | 6.86 |

### Example 3

### Shelf Life Stability

The accelerated aging test at elevated temperature was also used to evaluate the shelf life stability of the cyanoacrylate compositions packaged in the package system. The test can be performed in the oven at 80° C. for a period of 13 days. Based on ASTM F1980-2, 13 days accelerated aging at 80° C. correlates to 2 years of shelf life at ambient temperatures. The investigated compositions were tested for viscosity at different intervals of the aging process. As shown in Table 3, the viscosity of the cyanoacrylate composition in the package system after irradiation sterilization slightly increases as the accelerated aging proceeds but the increased viscosity of the aged samples at day 13 is so slight that it does not affect the performance of the cyanoacrylate composition or dispensing of the compositions from the packaging delivery system. The results demonstrate that the package systems are compatible with irradiation sterilization techniques so that cyanoacrylate compositions packaged inside can be sterilized via irradiation methods and provide long term stability of at least 2 years without adversely affecting the viscosity or performance of the cyanoacrylate compositions.

TABLE 3
The viscosity of composition 1, sterilized by irradiation at 11.8-
13.5 kGy, at different intervals of the accelerated aging at 80° C.

| Days | Viscosity | Viscosity | Viscosity | Average |
|---|---|---|---|---|
| in | Data #1 | Data #2 | Data #3 | Viscosity |
| Oven | (cPs) | (cPs) | (cPs) | (cPs) |
| 0 | 6.90 | 6.87 | 6.85 | 6.87 |
| 3 | 7.46 | 7.43 | 7.38 | 7.42 |
| 6 | 9.40 | 9.37 | 9.40 | 9.39 |
| 10 | 10.9 | 11.0 | 11.0 | 11.0 |
| 13 | 20.9 | 20.8 | 20.9 | 20.9 |

The extended shelf life stability of the cyanoacrylate compositions in the package system after irradiation sterilization was also evaluated by a real time assessment. The real time aging study was conducted at room temperature by keeping the cyanoacrylate compositions in the package system after irradiation sterilization in a designated environment where the temperature and humidity are monitored by a chart recorder. The temperature was controlled at 22° C.±5° C. and the humidity cannot exceed 80%. Viscosity, curing speed, and purity of the cyanoacrylate adhesive compositions in the package system after irradiation sterilization were evaluated at day 0, month 12, and month 24 or other intervals between day 0 and month 24 to assess the performance and stability of the adhesive compositions. The viscosity was used to evaluate the stability of the adhesive compositions. Compared to the cyanoacrylate adhesive compositions in the package system after irradiation sterilization at day 0, the viscosity of the cyanoacrylate compositions slightly increased as the real time/shelf life study proceeded. Table 4 shows an example of the real time shelf life study that is currently at month 19. The adhesive composition included 2-octyl cyanoacrylate with miscible polymerization accelerator. The adhesive composition was packaged in the ampoule and the overpack and underwent two sterilizations. As shown in Table 4, the viscosity of the cyanoacrylate composition at day 0 was 6.08 cPs, which is increased slightly to 9.48 cPs at month 19 of the real time aging indicating that the cyanoacrylate composition in the package system exhibits an extended shelf life.

TABLE 4
Viscosity of the sterilized cyanoacrylate composition by irradiation
in package system before and after the real time aging

| Time spent at room | |
|---|---|
| temperature | Average viscosity (cPs) |
| Day 0 | 6.08 |
| Month 6 | 6.69 |
| Month 9 | 7.16 |
| Month 12 | 7.16 |
| Month 15 | 8.05 |
| Month 19 | 9.48 |

The accelerated aging test of the cyanoacrylate compositions in the package system after irradiation sterilization can also be performed in the oven at 55° C. for a period of 85 days. 85 days at 55° C. is equal to about 2 years shelf life at room temperature. As compared to viscosity of the cyanoacrylate composition in the package system after irradiation sterilization at day 0, the viscosity of the cyanoacrylate composition at day 85 show essentially no change or slight increase in viscosity without affecting the performance of the adhesive composition. The aging test at 55° C. for 85 days also indicated an extended shelf life stability of at least 2 years of the cyanoacrylate compositions in the package system after irradiation sterilization methods.

### Example 4

### Setting Time Measurement

Pig skin (4×4 square inch) was prepared by wiping the surfaces of the skin with sterile gauze saturated with isopropanol. All oily substances were thereby removed from the pig skin. The surface was then wiped with sterile gauze to remove the isopropanol. The applicator containing cyanoacrylate was opened and adhesive was permitted to saturate the sponge applicator tip for about 10 seconds prior to application. A thin film was applied to the pig skin after which elapsed time was recorded by a stop watch. Set time was then recorded by stopping the clock when the film was dry as determined at the point where no liquid transfer occurred when the film was touched with a gloved finger. Two cyanocrylate compositions were packaged in the applicators and sterilized by E-beam sterilization at the dose range of 15-18 kGy. Composition 1 included 2-octyl cyanoacrylate as the main ingredient and a trace amount of polymerization initiator, 18-crown-6. Composition 1 also included BHA and SO₂ as the stabilizers. Composition 2 included 80% of 2-octyl cyanoacrylate and 20% of n-butyl cyanoacrylate. Composition 2 also included BHA and SO₂ as stabilizers. Table 3 shows the set time of these two cyanoacrylate compositions contained in the package system after E-beam sterilization.

TABLE 5
Set time measurement of two cyanoacrylate compositions
in the package system after E-beam sterilization

| | Set Time (Second) | | | | |
|---|---|---|---|---|---|
| | Test 1 | Test 2 | Test 3 | Test 4 | Average |
| Composition 1 | 21 | 14 | 17 | 16 | 17 |
| Composition 2 | 25 | 28 | 30 | 27 | 27.5 |

### Example 5

### Viscosity and Set Time

A 2-octyl cyanoacrylate composition was stored in the package system of an ampoule made of acrylonitrile-methyl acrylate copolymer and multi-layer seal foil, which were sealed tight onto the ampoule by heat. The multi-layer foil was acrylonitrile-methyl acrylate copolymer/ethylene acrylic acid copolymer/aluminum. The cyanoacrylate composition in the package system was sterilized by Gamma irradiation at the dose of 11.8-13.5 kGy. The package system also included the overpack, which was sterilized with the chemical sterilization. The stability of the composition was then evaluated by the accelerated aging at 55° C. for 85 days and the viscosity results are summarized in Table 6.

TABLE 6
The viscosity and set time of cyanoacrylate composition in the package
system, sterilized by irradiation at 11.8-13.5 kGy, at different
intervals of the accelerated aging at 55° C. for 85 days

| | Average | Average |
|---|---|---|
| | Viscosity | Set time |
| Days in Oven | (cPs) | (seconds) |
| 0 | 6.98 | 20 |
| 14 | 6.10 | 20 |
| 42 | 10.3 | 25 |
| 60 | 15 | 30 |
| 85 | 47 | 30 |

### Example 6

### Viscosity Before and after X-Ray Irradiation

A 2-octyl cyanoacrylate composition was sterilized by X-ray irradiation at two dose ranges of 6.96-8.59 and 11.89-13.69 kGy, respectively. The viscosity of the cyanoacrylate composition before and after Gamma sterilization is listed Table 7. The composition was housed in the ampoule and the overpack, and the package system underwent two sterilizations: irradiation and chemical sterilization.

TABLE 7
Viscosity of composition 1 before and after X-ray sterilization
at two different dose ranges

| | Viscosity (cPs) | | | | | | | |
|---|---|---|---|---|---|---|---|---|
| Dosage | | | | | After X-ray irradiation | | | |
| range | Before X-ray irradiation | | | | | | | aver- |
| (kGy) | 1 | 2 | 3 | average | 1 | 2 | 3 | age |
| 6.96-8.59 | 7.97 | 7.97 | 6.74 | 7.56 | 7.36 | 7.97 | 7.97 | 7.77 |
| 11.89-13.69 | | | | | 6.74 | 7.36 | 6.74 | 6.95 |

### Example 7

### Viscosity and Set Time

A 2-octyl cyanoacrylate composition was packaged in the ampoule/seal lid package system, which was sterilized by Gamma irradiation only at dose of 11.8-13.1 kGy. The ampoule and seal lid were sealed together at the temperature in the range of 150-155° C. under the pressure in the range of 2.3-2.5 bar. The stability of the composition was then evaluated by the accelerated aging at 80° C. for 13 days and the viscosity results were summarized in Table 8.

TABLE 8
The viscosity and set time of composition 1 in ampoule/lid
package system, sterilized by Gamma at 11.8-13.1 kGy,
at different intervals of the accelerated aging (AA) at 80° C.

| | | | | | Set | Set | Set | Set | |
|---|---|---|---|---|---|---|---|---|---|
| | | | | | Time | Time | Time | Time | Avg. |
| Days | Viscosity | Viscosity | Viscosity | Average | Date | Data | Data | Data | Set |
| in | Data #1 | Data #2 | Data #3 | Viscosity | #1 | #2 | #3 | #4 | Time |
| AA* | (cPs) | (cPs) | (cPs) | (cPs) | (Sec.) | (Sec.) | (Sec.) | (Sec.) | (Sec.) |
| 0 | 6.64 | 6.56 | 6.62 | 6.61 | 15 | 15 | 15 | 15 | 15 |
| 4 | 8.48 | 8.51 | 8.45 | 8.48 | 15 | 20 | 15 | 20 | 17.5 |
| 8 | 14.4 | 14.8 | 14.8 | 14.7 | 20 | 20 | 20 | 20 | 20 |
| 11 | 21.0 | 21.3 | 21.5 | 21.3 | 20 | 20 | 20 | 20 | 20 |
| 13 | 25.6 | 25.4 | 25.5 | 25.5 | 25 | 30 | 25 | 25 | 26.3 |

AA* represents accelerated aging.

### Example 8

### Sterility Test

The sterility of the cyanoacrylate compositions in the package was evaluated by the USP bacteriostasis and fungistasis test using the direct transfer method. The test samples were immersed into 500 ml of Soybean Casein Digest Medium (SCDM). The test microorganism, such as Bacillus subtilis, Candida albicans, and Aspergillus niger, was inoculated into each of the test sample containers and into a positive control container of the same medium at less than 100 colony forming units. All preparations were performed in an aseptic manner within a filtered clean bench. In order to obtain a quantitative measure of each microorganism, a duplicate plate count was performed. After inoculation, the test sample and positive control container were incubated at 20-25° C. for a five day maximum incubation period. Inoculated containers were observed periodically throughout the incubation period. Growth of the challenging microorganism was used to indicate the sterility. Cyanoacrylate compositions in the package system were sterilized by various irradiations and tested for sterility; no growth of the challenging microorganism was observed.

Although illustrated and described above with reference to certain specific embodiments and examples, the invention is nevertheless not intended to be limited to the details shown. Rather, various modifications may be made in the details within the scope and range of equivalents of the claims and without departing from the spirit of the invention. It is expressly intended, for example, that all ranges broadly recited in this document include within their scope all narrower ranges which fall within the broader ranges. In addition, features of one embodiment may be incorporated into another embodiment.

We claim:

1. A twice-sterilized package system including a stabilized cyanoacrylate monomer composition, the package system comprising:
an ampoule having sidewalls comprised of a material that is substantially impermeable to gas and substantially impermeable to moisture, which sidewalls surround a chamber containing a stabilized cyanoacrylate monomer composition comprising an amount of a free radical polymerization inhibitor and an anionic polymerization inhibitor effective to stabilize the cyanoacrylate monomer, and optionally, a polymerization accelerator mixed together with the cyanoacrylate monomer, which chamber comprises an opening that is sealed with a foil seal comprising a plurality of layers, wherein the ampoule containing the stabilized cyanoacrylate monomer composition has been sterilized by irradiation, thereby producing an ampoule containing a sterilized, stabilized cyanoacrylate monomer composition, and the sterilized, stabilized composition has a viscosity of about 3 cPs to about 12 cPs; and
an overpack, which is substantially permeable to gas, having a front wrapper and a back wrapper that are sealed together around the ampoule thereby housing the ampoule containing the sterilized, stabilized cyanoacrylate monomer composition, wherein the overpack housing the ampoule containing the sterilized, stabilized cyanoacrylate monomer composition has been sterilized by chemical sterilization, thereby producing the twice-sterilized package system including the sterilized, stabilized cyanoacrylate monomer composition having a viscosity of about 3 cPs to about 12 cPs;
wherein the twice-sterilized package system containing the sterilized, stabilized cyanoacrylate monomer composition, when stored in the package system for at least about two years, has a viscosity of less than about 50 cPs.

2. The package system of claim 1, wherein the sterilized, stabilized cyanoacrylate monomer composition, when stored in the package system for at least about two years, has a viscosity of about 10 cPs about 200 cPs.

3. The package system of claim 1, wherein the sidewalls of the ampoule have thickness of about 400 μm to about 3,000 μm.

4. The package system of claim 1, wherein the foil seal comprises an inner layer and one or more outer layers, and the inner layer comprises a material that is substantially impermeable to gas and moisture, and the material has a thickness of about 10 μm to about 100 μm.

5. The package system of claim 4, wherein the material is selected from the group consisting of poly(ethylene-co-alkyl acrylates), acrylonitrile polymers, acrylonitrile copolymers, polyamides, polyolefins, ethylene vinyl alcohol copolymers, ethylene-vinyl acetate copolymer, ethylene-alkyl acrylate-acrylic acid terpolymer, ethylene acrylic acid copolymer, polychlortrifluoethylene, and combinations thereof.

6. The package system of claim 4, wherein the one or more outer layers of the foil seal each independently have a thickness of about 5 μm to about 200 μm.

7. The package system of claim 4, wherein the one or more outer layers of the foil seal comprise polyethylene (PE), polystyrene (PS), aluminum foil, polypropylene (PP), polycarbonate (PC), ethylene acrylic acid copolymer (EAA), polyethylene terephthalate (PET), polystyrene (PS), thermoplastic elastomer (TPE), polytetrafluoroethylene (PFTE), polyvinylchloride (PVC), or a combination thereof.

8. The package system of claim 1, wherein the front wrapper and back wrapper of the overpack comprise a blister around the ampoule.

9. The package system of claim 1, wherein the free radical polymerization inhibitor is selected from the group consisting of butylated hydroxy anisole (BHA), catechol, butylated hydroxytoluene (BHT), 4-ethoxyphenol, 2-tert-butyl-4-methoxyphenol, methoxyphenol, and mixtures thereof.

10. The package system of claim 1, wherein the anionic polymerization inhibitor is selected from the group consisting of sulfur dioxide, boron trifluoride, sulfonic acids, sultones, alkylsulfone, lactones, sulfuric acid, phosphorous acids, carboxylicacids, acetic acid, alkyl sulfoxide, alkyl sulfate, alkyl sulfide, hydrofluoric acid, and mixtures thereof.

11. The package system of claim 1, wherein the composition includes a polymerization accelerator, and the polymerization accelerator is selected from the group consisting of calixarenes, oxacalixarenes, silacrowns, crownethers, cyclodextrin and its derivatives, polyethers, aliphatic carboxylic acid esters, alkyl and aryl amine derivatives, polyalkylene oxides and derivatives, and mixtures thereof.

12. The package system of claim 1, wherein the stabilized cyanoacrylate monomer composition further comprises a plasticizing agent.

13. The package system of claim 1, wherein the stabilized cyanoacrylate monomer composition further comprises a thickening agent.

14. The package system of claim 1, wherein the cyanoacrylate monomer comprises 2-ocytl cyanoacrylate or a mixture of 2-octyl cyanoacrylate and n-butyl cyanoacrylate.

15. The package system of claim 1, wherein the irradiation comprises Electron beam radiation, gamma radiation, or X-rays.

16. The package system of claim 1, wherein the chemical sterilization comprises ethylene oxide sterilization.

17. The package system of claim 1, wherein the overpack comprises an applicator for dispensing the sterilized, stabilized cyanoacrylate monomer composition, the applicator comprising a foam or sponge tip, and optionally comprising a reservoir.

18. The package system of claim 17, wherein the ampoule material is flexible and capable of being squeezed to release the sterilized, stabilized cyanoacrylate monomer composition from the chamber through the foil seal or through the connection between the sidewalls and the foil seal.

19. The package system of claim 1, wherein the package system is produced by a process comprising the following steps:
(a) inserting a stabilized cyanoacrylate monomer composition into the chamber of an ampoule comprising sidewalls comprised of a material that is substantially impermeable to gas and substantially impermeable to moisture, which sidewalls surround a chamber having an opening;
(b) sealing the opening of the chamber with a foil seal comprising a plurality of layers, thereby containing the composition within the chamber;
(c) sterilizing the ampoule containing the composition by irradiation;
(d) surrounding the sterilized ampoule with a front wrapper and a back wrapper and sealing the front wrapper and back wrapper together to form an overpack; and,
(e) sterilizing the overpack containing the ampoule containing the composition by chemical sterilization.

20. The package system of claim 19, wherein the chemical sterilization of step (e) comprises ethylene oxide sterilization.

21. The package system of claim 19, wherein the ampoule and the foil seal are heat-sealed together under a temperature and pressure after the stabilized cyanoacrylate monomer composition is inserted into the ampoule.

22. The package system of claim 21, wherein the temperature used to seal the plastic ampoule and the multi-layer foil seal is in the range of about 110° C. to about 170° C.

23. The package system of claim 21, wherein the pressure used to seal the ampoule and the multi-layer foil seal is in the range of about 1 bar to about 15 bar.
